(19) United States
(12) Patent Application Publication
(10) Pub. No.: US 2004/0106838 A1
(43) Pub. Date: Jun. 3, 2004

(54) Use of ionic liquids to separate diolefins

(75) Inventors: Ronald Scott Smith, Calgary (CA); Patricio S. Herrera, Calgary (CA); Jeffrey Sean Reynolds, Calgary (CA); Andrzej Krzywicki, Calgary (CA)

Correspondence Address: KENNETH H. JOHNSON, P.O. BOX 630708, HOUSTON, TX, 772630000

(73) Assignee: NOVA Chemicals (International) S.A.

(21) Appl. No.: 10/308,307

(22) Filed: Dec. 2, 2002

(51) Int. Cl. C07C 7/00

(52) U.S. Cl. 585/862; 585/860

(57) ABSTRACT

The present invention relates to the separation of diolefins from mixed streams of hydrocarbons using ionic liquids in the absence of metal compounds.

### FIELD OF THE INVENTION

The present invention relates to the separation of diolefinic alkenes from alkanes and/or mono-olefinic alkenes using an ionic liquid in the absence of metal compounds.

### BACKGROUND OF THE INVENTION

In the cracking of feedstocks to produce alkenes the resulting product is typically a mixture of various alkenes, such as ethylene, propylene, and butadiene, and various alkanes or paraffins such as ethane, propane, and higher alkanes. With close boiling products such as butadiene, butenes, and butanes it is necessary to separate the products and co-products using distillation or extractive distillation methods. The distillation may be carried out at low temperatures or may be carried out using higher pressures and corresponding higher temperatures.

It is known to separate olefins and diolefins from paraffins by forming complexes with metals such as silver or copper. The resulting copper or silver complex is preferentially soluble in a liquid not miscible or soluble in the paraffin, such as water. The streams are separated and then the olefin/diolefin is released from the complex typically by a temperature or pressure change. The regenerated metal compound is then capable of being reused to complex more olefin. In some cases the metal compound is adsorbed or complexed on the surface of an ion exchange resin or in a membrane separation film and the olefin is separated from the alkane. Representatives of such art include Canadian Patent 1,096,779 issued Mar. 3, 1981 to Deutsche Texaco A.G.; U.S. Pat. No. 3,979,280 issued Sep. 7, 1976 and assigned to Deutsche Texaco A.G.; U.S. Pat. No. 4,328,382 issued May 4, 1982 assigned to Erdoelchemie G.m.b.H.; and U.S. Pat. No. 3,441,377, issued Apr. 29,1969 to ESSO Research and Engineering Co.

Most recent in this line of technology is U.S. Pat. No. 6,339,182 B1 issued Jan. 15, 2002 to Munson et al., assigned to Chevron U.S.A. Inc. This patent teaches the absorption of alkenes by metal salts, typically silver or copper salts in ionic liquids. The alkenes are typically initially present as an admixture with paraffins. The alkenes are regenerated by separation from the metal complex by temperature or pressure change or application of an entrainment gas such as an inert gas.

The present invention is distinct over the above art as it does not require the presence of a metal complex. Applicants have discovered that olefins are preferentially soluble in some ionic liquids without the presence a metal (e.g. silver or copper) salt. The present invention is further distinct over the above art in that it preferentially separates di-olefins from corresponding mono-olefins.

The present invention seeks to provide a simple process for the separation of diolefins from other hydrocarbons, particularly alkanes and mono-olefins.

### SUMMARY OF THE INVENTION

The present invention provides a process for separating one or more members selected from the group consisting of C₄₋₈ diolefins which are unsubstituted or substituted by up to three C₁₋₄ alkyl radicals from a mixture comprising at least one of said diolefins and at least one other hydrocarbon comprising contacting said mixture with a nitrogen containing ionic liquid having a melting temperature below 80° C. to preferentially take said one or more diolefins into said ionic liquid, separating said ionic liquid from said at least one other hydrocarbon and regenerating said ionic liquid and releasing said at least one diolefin.

### DETAILED DESCRIPTION

In accordance with the present invention the one or more diolefins selected from the group consisting of C₄₋₈ conjugated diolefins (or dienes) may be separated from one or more hydrocarbons, typically mono-olefins and paraffins, typically having up to about 20 carbon atoms, preferably C₁₋₁₈ paraffins and C₂₋₁₈ mono-olefins.

The diolefins are typically C₄₋₈ diolefins. The diolefins may be conjugated or non-conjugated. Some diolefins include butadiene, including 1,3-butadiene, hexadiene including 1,4-hexadiene and 1,5-hexadiene, and octadiene including 1,7-octadiene. The dienes may be substituted by a C₁₋₄ alkyl radical such as isoprene. Preferably the dienes are hydrocarbyl compounds and do not contain other atoms or functional groups.

The olefins may be selected from the group consisting of alpha-olefins including ethylene, propylene, butene, hexene, and octene, preferably ethylene, butene, hexene, and octene. The olefins may be other than alpha-olefins such as 2-methyl-2-butene. Preferably the olefins are hydrocarbyl olefins and do not contain other atoms or functional groups.

The paraffins may be selected from the group consisting of C₁₋₂₀, preferably C₁₋₁₈ paraffins, most preferably C₂₋₁₀ paraffins. Such paraffins include propane, butane, pentane, hexane, heptane, octane, nonane and decane which are unsubstituted or may be substituted by one or more C₁₋₄ alkyl radicals.

The mixtures to be treated in accordance with the present invention may be subject to a number of treatments prior to being contacted with the ionic liquid. Such treatments are well known to those skilled in the art and include for example removal of non-hydrocarbon species (e.g. CO, CO₂ and water) and hydrogenation such as hydrogenation of acetylenes.

Ionic liquids are organic compounds that are liquid at room temperature. They differ from most salts, in that they have very low melting points. They tend to be liquid over a wide temperature range and have essentially no vapor pressure. Most are air and water stable, and they are used in accordance with the present invention to solubilize diolefins. The properties of the ionic liquids can be tailored by varying the cation and anion. Examples of ionic liquids are described, for example, in J. Chem. Tech. Biotechnol., 68:351-356 (1997); Chem. Ind., 68:249-263 (1996); and J. Phys. Condensed Matter, 5:(supp 34B):B99-B106 (1993); Chemical and Engineering News, Mar. 30, 1998, 32-37; J. Mater. Chem., 8:2627-2636 (1998); and Chem. Rev., 99:2071-2084 (1999), the contents of which are hereby incorporated by reference.

Many ionic liquids are formed by reacting a nitrogen-containing heterocyclic ring, preferably a heteroaromatic ring, with an alkylating agent (for example, an alkyl halide) to form a quaternary ammonium salt, and performing ion exchange or other suitable reactions with various counter ions such as Lewis acids or their conjugate bases to form ionic liquids (nitrogen based ionic liquid). Examples of suitable heteroaromatic rings include substituted pyridines, imidazole, substituted imidazole, pyrrole and substituted pyrroles. These rings can be alkylated with virtually any straight, branched or cyclic C₁₋₂₀ alkyl group, but preferably, the alkyl groups are C₁₋₁₆ groups, since groups larger than this tend to increase the melting point of the salt.

Ionic liquids have also been based upon various triarylphosphines, thioethers, and cyclic and non-cyclic quaternary ammonium salts. Counter-ions which have been used include chloroaluminates, bromoaluminates, gallium chloride, tetrafluoroborate, tetrachloroborate, hexafluorophosphate, nitrate, trifluoromethane sulfonate, methylsulfonate, p-toluenesulfonate, hexa fluoroantimonate, hexa fluoroarsenate, tetrachloroaluminate, tetrabromoaluminate, perchlorate, hydroxide anion, copper dichloride anion, iron trichloride anion, zinc trichloride anion, as well as various lanthanum, potassium, lithium, nickel, cobalt, manganese, and other metal-containing anions.

In accordance with the present invention the organic portion of the ionic liquid is typically a nitrogen containing C₅₋₈ heterocyclic aromatic compound. The heterocyclic aromatic compound may be unsubstituted or substituted by up to three C₁₋₆, preferably C₁₋₄ alkyl radicals. The heterocyclic aromatic compound may be selected from the group comprising pyrrolium, imidazolium, and pyridinium which are unsubstituted or substituted by up to two C₁₋₄ alkyl radicals, for example 1-butyl-3-methylimidazolium.

Useful counter ions include borate compounds, preferably tetrahaloborates most preferably tetrafluoroborate (the corresponding acid form of Lewis acid would, for example, be H⁺BF₄⁻). Other counter-ions which may be suitable for use in the present invention are discussed in U.S. Pat. No. 6,339,182.

Some ionic liquids which may be used in accordance with the present invention include 1-butyl-3-methylimidazolium tetrafluoroborate and 1-hexyl-3-methylimidazolium tetrafluoroborate. Further or differently substituted homologues of these compounds are within the scope of the present invention. Other ionic liquids would be apparent to those skilled in the art.

The ionic liquid may optionally contain from 0 up to about 15, preferably less than 10% by volume of water.

The above noted diolefinic hydrocarbons can be selectively separated from mixtures containing one or more of such compounds and other hydrocarbons such as paraffins, olefins, and mixtures thereof. The separation involves contacting the mixture containing one or more of the diolefins with the ionic liquid. The ionic liquid takes up the diolefins present in the mixture. The ionic liquid is then separated from the mixture (which is free from or has a significantly reduced content of such diolefinic compounds). The hydrocarbon stream can be separated from the ionic liquid using conventional means including, for example, decantation, and the like. In the separation of the residual hydrocarbon stream from the ionic liquid care needs to be taken not to subject the ionic liquid to conditions that will cause it to give up the one or more of the diolefins.

The mixture containing one or more of the diolefins may be contacted with the ionic liquid using well known methods including, co-current, counter-current, or staged in stirred tanks. Counter-current methods are preferred. The mixture containing one or more diolefins can be in the gas phase or the liquid phase. The ionic liquid will be in the liquid phase. Typically the contact will take place at temperatures less than about 80° C., preferably less than 50° C., more preferably about room temperature (i.e. from 15° C. to 25° C.). The pressure may be low (i.e. up to 1,000 psig (6,895 kPa), preferably less than 100 psig (689.5 kPa). If the contact with the ionic liquid is under pressure the pressure on the ionic liquid should not be reduced until it is desired to release the one or more diolefins from the ionic liquid.

The one or more of the diolefins may then be recovered from the ionic liquids using a number of regeneration techniques. These techniques may include thermal regeneration (increasing the solution temperature to release the diolefins), pressure swing regeneration (reducing the pressure), and combinations thereof. Entrainment gasses, typically inert gasses, preferably nitrogen may also be passed through the ionic liquid to entrain and release the diolefins from the ionic liquid. Entrainment gasses may be used with either or both of the foregoing techniques to release the diolefins from the ionic liquid.

Release of the one or more diolefins may be carried out in a packed tower or flash drum, preferably a packed tower generally by using a combination of increased temperature and lower pressure. The temperatures may range from about 10° C. to about 15° C., (although higher temperatures may be required for relatively high molecular weight diolefins) preferably from about 120° C. to about 140° C., and the pressure may range from vacuum pressures to about 50 psig (345 kPa), preferably from about 10 psig (about 68.9 kPa) to about 30 psig (about 207 kPa). The temperatures should be higher and the pressures should be lower for higher molecular weight diolefins. The decomposition temperature of the ionic liquids should not be exceeded.

The packed tower or flash drum may include multi-stage stripping or flashing for increased energy efficiency. In such systems, the ionic solution rich in one or more diolefins is flashed and stripped at progressively higher temperatures and/or lower pressures. The design of such systems is well known to those skilled in the art.

Conventional heating means known to those of ordinary skill in the art, including steam and preferably low pressure steam, may be used to release the one or more diolefins from the ionic liquid. One inexpensive heat source in the lower end of the temperature range is quench water. The packed column or flash drum is preferably equipped with a water wash section in the top to prevent entrainment of the desorbed gases.

The ionic liquid solution can then be removed from the bottom of the packed column or tower or flash drum and recycled back to the contact device.

The present invention provides a simple and relatively cheap means to separate butadiene from butene and butane products from an ethane cracker, ethane/propane cracker, or flexi-cracker. Another commercial use is in the separation of isoprene from mixed C₅ co-product streams in petrochemical plants.

The present invention will now be illustrated by the following non-limiting examples in which unless otherwise indicated weight is in grams and parts is parts by volume.

### EXAMPLE 1

### C₅ Hydrocarbon Solubility by Ionic Liquids

The present example investigated the solubility of paraffin, olefin, and diolefin C₅ hydrocarbons, isopentane, 2-methyl-2-butene, and isoprene respectively, in 1-butyl-3-methylimidazolium tetrafluoroborate (bmim⁺ BF₄⁻) and demonstrates the corresponding selectivity for isoprene over the corresponding paraffin (isopentane) and the corresponding olefin (2-methyl-2-butene). The testing apparatus consisted of a flat-bottomed florence flask with a graduated neck. The flask was charged with 75 mL of bmim⁺ BF₄⁻, the level being recorded. A known quantity of a C₅ hydrocarbon was then added to the flask and the flask was sealed. The overall liquid level and the location of the liquid-liquid interfacial meniscus were recorded. The mixture was then agitated to contact the two liquids and the two phases were allowed to separate. The locations of all meniscuses were then recorded. Agitation and phase separation was then repeated until the liquid levels remained unchanged. The volume change of the hydrocarbon phase corresponds to the quantity of hydrocarbon dissolved in the ionic liquid. The testing was conducted at ambient temperature. The results are summarized in Table 1.

TABLE 1
C₅ Solubility in bmim⁺ BF₄⁻ Ionic Liquid

| | Solubility | | |
|---|---|---|---|
| | (mol C₅/L | Selectivity | Selectivity |
| | Ionic | Over | Over |
| Hydrocarbon | Liquid) | Isopentane | 2-Methyl-2-Butene |
| Isopentane | 0.034 | — | — |
| 2-Methyl-2-Butene | 0.360 | 10.6 | — |
| Isoprene | 0.611 | 18.0 | 1.7 |

### EXAMPLE 2

### Liquid-Liquid Extraction of Butadiene by Ionic Liquids

The present example demonstrates the selectivity of 1-butyl-3-methylimidazolium tetrafluoroborate (bmim⁺ BF₄⁻) for butadiene over C₄ mono-olefins and paraffins and room temperature. The testing apparatus consists of three sample cylinders (150 cc, 10 cc, and 50 cc) connected by ⅜-inch tubing (in the order listed) with valves allowing for isolation of each cylinder. A gas syringe is used to take C₄ samples for GC analysis. Various mixed C₄S were used which contained varying quantities of 1,3-butadiene (ranging from 15-85 weight %). The composition range of the non-butadiene fraction of the standards was (in weight %) n-butane 29-33%, 1-butene 20-33%, isobutene 0-36%, trans-2-butene 7-17%, and cis-2-butene 5-17%.

To measure C₄ extraction, 40 mL of bmim⁺ BF₄⁻ absorbent is loaded into the 150 cc cylinder and the 10 cc and 150 cc cylinders are then evacuated. The 10 cc cylinder is then isolated and charged with mixed C₄ liquid. This liquid is then added to the 150 cc cylinder. The 10 cc cylinder is then isolated and a second 10 cc is then added to the and subsequently added to the 150 cc cylinder and the 150 cc cylinder is isolated. The apparatus is then agitated to maximize contact between the C₄ and ionic liquid phases within the 150 cc cylinder. Following agitation, the liquid is allowed to sit 12 hours to allow the two liquid phases to separate.

To determine the level and composition of C₄ in the ionic liquid, a 10 cc sample of ionic liquid from the 150 cc cylinder is charged into the 10 cc cylinder. The 150 cc cylinder is then isolated and the 10 cc ionic liquid sample is charged to the 50 cc cylinder, which is then isolated. The 50 cc cylinder is then heated and the gas syringe is used to collect C₄ gas evolving from the ionic liquid. The collected gas is analyzed by GC.

These pseudo-equilibrium extraction experiments conducted for different butadiene concentrations yielded enhanced concentrations of butadiene in the C₄ extracted by the ionic liquid. Table 2 summarizes the findings.

TABLE 2
Butadiene Extraction by bmim⁺BF₄⁻Ionic Liquid

| Butadiene Concentration | Butadiene Concentration |
|---|---|
| in Feed (wt %) | in Extracted C₄ (wt %) |
| 15 | 37.6 |
| 30 | 43.3 |
| 50 | 74.1 |
| 70 | 82.0 |
| 85 | 91.8 |

What is claimed is:

1. A process for separating one or more members selected from the group consisting of C₄₋₈ diolefin hydrocarbons which are unsubstituted or substituted by up to three C₁₋₄ alkyl radicals from a mixture comprising at least one of said diolefins and at least one other hydrocarbon selected from the group consisting of C₁₋₁₈ paraffins and mono-olefins comprising contacting said mixture with a nitrogen containing ionic liquid having a melting temperature below 80° C. to preferentially take said one or more diolefins into said ionic liquid, separating said ionic liquid from said at least one other hydrocarbon and regenerating said ionic liquid and releasing said at least one diolefin.

2. The process according to claim 1, wherein in said ionic liquid the organic component is a heterocyclic nitrogen-containing aromatic compound.

3. The process according to claim 2, wherein said heterocyclic nitrogen containing aromatic compound is a C₅₋₈ nitrogen containing aromatic compound which is unsubstituted or substituted by up to three C₁₋₆ alkyl radicals.

4. The process according to claim 1, wherein said nitrogen-based ionic liquid is a nitrogen containing tetrafluoroborate ionic liquid.

5. The process according to claim 4, wherein said ionic liquid is selected from the group consisting of imidazolium ionic liquids which are unsubstituted or substituted by up to two C₁₋₄ alkyl radicals.

6. The process according to claim 5, wherein said ionic liquid is 1-butyl-3-methylimidazolium tetrafluoroborate.

7. The process according to claim 6, wherein said mixture is in the gas or liquid state.

8. The process according to claim 7, wherein said regeneration of ionic liquid and said releasing of at least one member is effected using one or more treatments selected from the group consisting of increasing temperature, decreasing pressure, and passing an entraining gas through said ionic liquid.

9. The process according to claim 8, wherein said mixture and said ionic liquid are contacted in a counter-current flow.

10. The process according to claim 8, wherein said mixture and said ionic liquid are contacted in co-current flow.

11. The process according to claim 8, wherein said mixture and said ionic liquid are contacted in a continuous stirred tank reactor.

12. The process according to claim 1, wherein in the ionic liquid optionally contains from 0 to 15 volume % of water.

13. The process according to claim 5, wherein in the ionic liquid optionally contains from 0 to 15 volume % of water.

14. The process according to claim 6, wherein in the ionic liquid optionally contains from 0 to 15 volume % of water.
